(19) United States
(12) Patent Application Publication
(10) Pub. No.: US 2007/0239013 A1
(43) Pub. Date: Oct. 11, 2007

(54) Delay controller for ultrasound receive beamformer

(75) Inventor: Radu Alexandru, Cheshire, CT (US)

Correspondence Address: BACHMAN & LAPOINTE, P.C., 900 CHAPEL STREET, SUITE 1201, NEW HAVEN, CT, 06510, US

(73) Assignee: Aloka Co., Ltd.

(21) Appl. No.: 11/387,646

(22) Filed: Mar. 22, 2006

(51) Int. Cl. A61B 8/00

(52) U.S. Cl. 600/443

(57) ABSTRACT

An ultrasound system includes an ultrasound transducer having an array of elements and a beam origin located between two adjacent elements, each of said elements for converting received energy into an echo signal; and a beamformer includes at least one channel having delay circuitry and tracking circuitry for tracking an ultrasound beam; a summer for summing phased-aligned signals to form a beam; and an initialization controller having initialization controller circuitry for calculating initialization parameters starting from a reduced parameter table according to a process for calculating initialization parameters.

### FIELD OF THE INVENTION

The present invention relates to ultrasound imaging. More specifically, the invention relates to the focusing of received ultrasound beams.

### BACKGROUND OF THE INVENTION

In ultrasound imaging, a transducer is used to transmit ultrasound beams into the medium to be examined, for example, a region of the human body; receive the ultrasound echoes reflected from various discontinuities in the medium; and, transform the reflected ultrasound echoes into electrical signals. The electrical signals then undergo a number of processing steps and are eventually transformed into an image which can be displayed on a device such as a cathode ray tube or printed in order to be examined by a physician.

Ultrasound transducers typically consist of arrays of small rectangular piezoelectric elements. A subset of such elements used to transmit or receive an ultrasound beam is called a transmit or receive aperture, respectively. Typically, the geometrical centers of transmit and receive apertures coincide, and the ultrasound beam(s) are represented as linear beam axes originating at the center of the apertures.

The receive operation is performed by a multi-channel receive beamformer. The multi-channel receive beamformer applies delays and weights to the signals received by various receive aperture elements and sums them to obtain focused signals along the desired beam axis. The purpose of the delays is to compensate for the difference in arrival time caused by difference in propagation paths from the point of interest of the medium to the different elements of the aperture. In order to obtain ultrasound beams focused at multiple depths along the beam axis, the receive delays are varied with depth such that all the signals which are summed to obtain the echo from a point on the beam axis arrive from that same point. This is called dynamic receive focus, and the image quality is critically dependent on the accuracy of the dynamic receive delays or equivalently of the echo arrival time. It is known in the art that a delay accuracy of 1/32 F_{c} is desirable, where F_{c} is the center frequency of the transducer's frequency characteristic.

The received signals can be delayed by various processes, both analog and digital, but in all cases a delay controller, or arrival time controller, has to produce the desired delay control signals. Practical beamformers use circuits which calculate either the dynamic delay or the arrival time in real time starting from a small number of pre-calculated initialization parameters. One such circuit of the prior art uses a piecewise linear approximation of either arrival time or delay curve, where the depth range is divided into several segments defined by their start and end points, or equivalently by their start point and their segment length, and within each segment the quantity of interest is approximated by a linear function with a certain slope. Experience has shown that for the imaging geometries used in phased array medical ultrasound eight segments are sufficient to achieve the approximation with a maximum error of approximately 1/32 F_{c}. Eight segments require sixteen (16) initialization parameters per element for each beam direction. For a typical phased array of 128 elements and 128 beam directions, after reducing the number of parameters by a factor of 2 using the symmetry of the beams, the required parameter memory size is 128 Kwords. This required parameter memory size may represent a considerable cost especially in low-end portable systems or in other compact designs where space and power are scarce, and has other disadvantages such as long dead time when switching from one probe to another which requires loading new parameter tables.

Therefore, a process is needed to reduce the number of initialization parameters for piecewise linear approximation delay controllers.

### SUMMARY OF THE INVENTION

In accordance with one aspect of the present invention, in an ultrasound imaging system comprising an ultrasound transducer having an array of elements and a beam origin located between two adjacent elements, each element for converting received energy into an echo signal, a process for calculating initialization parameters from a reduced parameter table, the process broadly comprising generating a piecewise linear approximation of a timing function of a reflected ultrasound beam for a first left element located to the left of a beam origin and a first right element located to a right of a beam origin; generating at least one initialization parameter comprising an initial time, an integer-valued segment start, an integer-valued segment end and a slope of a segment for said piecewise linear approximation for the first left element and the first right element; selecting a maximum acceptable error proportional to a distance measured from the beam origin to a first left center of the first left element or to a first right center of the first right element; and generating at least one next initialization parameter comprising a next initial time, a next integer-valued segment start, a next integer-valued segment end, and a next slope of at least one next segment for the piecewise linear approximation for at least one adjacent left element to the left of the first left element and at least one adjacent right element to the right of the first right element.

In yet another aspect of the present invention, an ultrasound system broadly comprises an ultrasound transducer broadly comprising an array of elements and a beam origin located between two adjacent elements, each of the elements for converting received energy into an echo signal; and a beam former broadly comprising an initialization controller including initialization controller circuitry for calculating initialization parameters according to the process of claims 1-20; at least one channel including delay circuitry and tracking circuitry for tracking an ultrasound beam; and a summer for summing phase-aligned signals to form a beamformed signal.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a representation of a receive beamformer;

FIG. 1a is a representation of a general block diagram of a beamformer channel;

FIG. 2 illustrates an imaging geometry used to calculate an echo arrival time in a phased array imaging;

FIG. 3a shows the relationship between depth along the beam axis and arrival time to elements of the echo from the respective depth;

FIG. 3b shows a segment of the arrival time curve and its linear approximation;

FIG. 4 shows a delay controller circuit for use in the delay controller of FIG. 1a; and

FIG. 5 shows an initialization controller circuit for the initialization controller of FIG. 1.

Like reference numbers and designations in the various drawings indicate like elements.

### DETAILED DESCRIPTION OF THE INVENTION

By choosing a segment end-point proportional with an element's distance from a beam origin of the phase array, the segment slopes become common to all elements to the left and all elements to the right of the beam origin, and the segment end-points for each element to the left and each element to the right of the beam origin can be calculated from only one set of left end-points and one set of right end-points, respectively. The use of only one set of left end-points and one set of right end-points results in a reduction of the number of initialization parameters by a factor equal to half of the number of elements in the phase array. In the instant example, the parameter table size decreases from 128 Kwords for a conventional phase array having 128 elements and 128 beam directions to only 2 Kwords for the same conventional phase array. The same process, and system employing said process, may also be applied to constructing a parameter table where end-points are substituted for segment lengths.

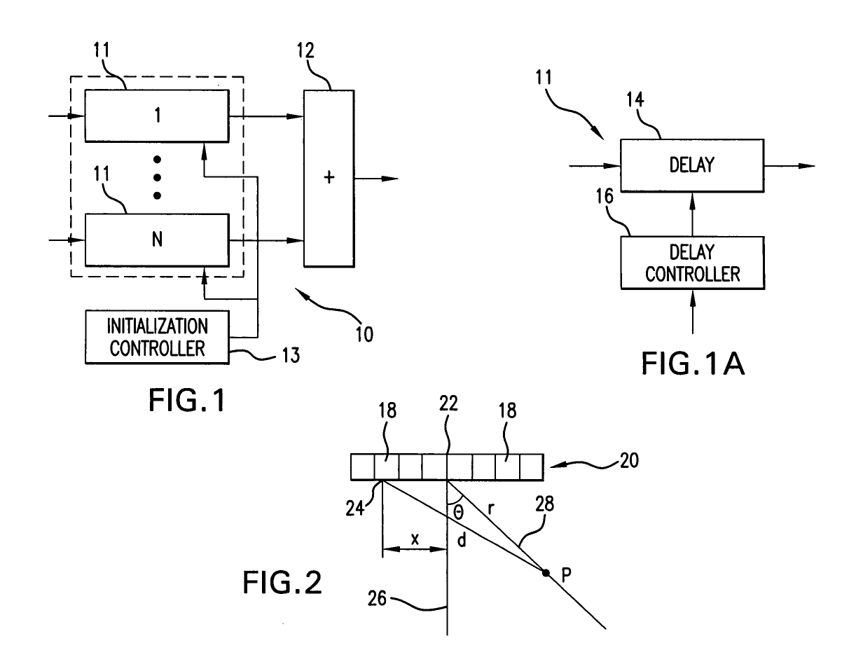

Referring now FIGS. 1-1a, a representation of a receive beamformer 10 for use in an ultrasound system is shown. Receive beamformer 10 may include at least one channel 11 (to the N^{th} channel 11), a summer 12 and an initialization controller 13. The channel 11 may include a delay block 14 and a delay controller 16. Before a beam may be transmitted, the initialization controller 13 may initialize the delay controller 16 of each channel 11 according to the beam characteristics, e.g. steering angle; array geometry and beam position within the aperture of each element 18 connected to the channel 11. After the beam is transmitted, echoes may begin arriving at the array elements 18, may each be converted to electrical signals, and may be preprocessed, e.g. amplified and filtered, and may be fed to each input of each channel 11. The delay blocks 14 may further process and delay the signals under the control of delay controllers 16, such that the signals may arrive phase-aligned at the inputs of the summer 12. The phase-aligned signals may then be summed to form a beamformed signal.

Delay controller 16 may utilize a piecewise linear approximation technique as known to one of ordinary skill in the art, which may vary slightly depending upon the particular process of implementing the delay block 14. For example, the delay block 14 may include an arrangement of analog delay lines (not shown) and analog multiplexers (not shown) to provide a coarse delay, combined with analog mixers (not shown) to provide a fine phase delay. In another example, the delay block 14 may include an analog-to-digital converter (not shown) to convert the electrical signal into its numerical representation, a memory storage device (not shown) which provides coarse delay at the resolution of the sampling period, and an interpolator (not shown) to provide fine delay, e.g., fine subsample delay. In yet another example, an analog-to-digital converter's clock (not shown) may be phase-modulated to provide the fine delay and the digital signals are then written to a memory storage device (not shown) which provides the coarse delay. The control signals that may be generated by delay controller 16 are generally one of three types: delay (δ), arrival time (t) or the sign of a decision quantity indicating that the delay has to be reduced by a fraction of the clock period. The system and process of the present invention will be explained with reference to the arrival time (t); the other two types of control signals may be easily derived from the system and process as described herein by one of ordinary skill in the art. The present invention is incorporated in the initialization controller 13 which provides the parameters for at least one delay controller 14 of at least one channel 11.

Referring now to FIG. 2, an ultrasound transducer generally comprises a phased array 20 having a center element 22, or array center O, and at least one element 18 to the left of the center element 22 and to the right of the center element 22. Each element 18 includes a center 24 located at a distance x from the center element 22. Since phased arrays typically possess an even number of elements and the true center falls between two elements 18, the center element 22 is theoretical and used for the purpose of explanation. A vertical axis 26 is shown for the purpose of providing a frame of reference, e.g., a Cartesian coordinate system. Theoretically, an ultrasound beam may originate from the array center 0, that is, the beam origin, and travel a distance r to a point P as shown in FIG. 2. The ultrasound beam may then reflect off a media (not shown) and travel a distance d to the center 24 of an element 18. The center 24 of element 18 may be situated at a coordinate x relative to the vertical axis 26. The ultrasound beam from array center O to point P may be referred to as a beam axis 28, or beam axis OP. The beam axis OP may be steered at an angle θ relative to the vertical axis 26. In this example, the value of x would be negative. Using the distances r and d and the angle 0, the arrival time of an echo from point P on the beam axis 26 to element 18 of the phased array 20 may be calculated by one of ordinary skill in the art.

The total travel time, or the arrival time, of the ultrasound beam through the media is shown below in Equation (1):
t=(r+d)/c (1)
where t is the total travel time of the ultrasound beam; r is the distance from the array center aperture to point P; d is the distance from point P to an element's center; and, c is the velocity of the ultrasound beam.

For beamforming applications, t has to be evaluated for discrete values of r at intervals Δr using an Equation (2) as follows:
Δr=cT/2 (2)
where Δr corresponds to the 2-way travel time along the beam axis 26 between two sampling times; T is a unit of time; and, c is the velocity of the ultrasound beam.

We choose T=1/F as a unit of time, where T is the sampling period and F is the sampling frequency and cT/2 is a unit of distance the ultrasound beam travels in both directions, i.e., forward and backward (reflection), in an amount of time T. With these units r takes integer values n=0, 1, . . . and t takes the values shown in Equations (3) and (4) as follows:
tₙ=(n+dₙ)/2 (3)
where
dₙ=\sqrt{n²+x²−2n×sin θ} (4)
wherein tₙ is the arrival time; n is the distance from the array center aperture to point P as measured as an integer value; dₙ is the distance from point P to an element center; and, x is the distance from the center of an element to the aperture of the array center element measured in units of cT/2.

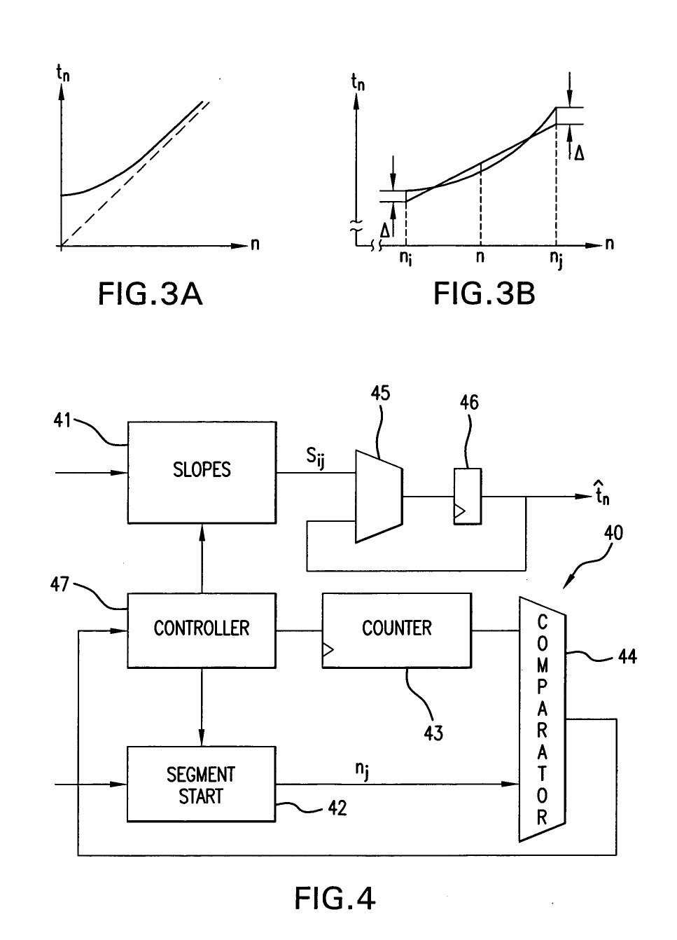

Referring now to FIG. 3a, the graph shows the relationship between the depth n along the beam axis 28 and the arrival time tₙ for two elements 18 is illustrated. The dashed line corresponds to an element at the center 22 of the aperture and the continuous line to an element 18 away from the center element 22. For the center element 22 whose center is at the theoretical origin of the beam axis 28, tₙ=n varies linearly with n with a slope S=1, such that tₙ increases by 1 when n increases by 1. For the element 18, t initially has a slope S less than 1, and the slope gradually increases with depth and approaches 1, i.e., S=A. Referring now to FIG. 3b, a portion (segment) situated between the depths nᵢ and nⱼ of the tₙ curve is shown for an element 18 away from the center element 22, and its linear approximation \hat{t}ₙ is calculated according to Equation (5):
\hat{t}ₙ=tₙᵢ−Δ+(n−nᵢ)Sᵢⱼ (5)
where Δ is the absolute value of the maximum acceptable error and Sᵢⱼ is the segment's slope as represented by the Equation (6):
Sᵢⱼ=(tₙⱼ−tₙᵢ)/(nⱼ−nᵢ) (6)

With these formulas the estimate error at the end of the segment, that is, the start of next segment, has the same value −Δ as at the start of the segment. When implementing the process of the present invention, the choice of error −Δ at the two endpoints of the segments may be made possible by the concave shape of the arrival time curve. This choice of error may facilitate the initial calculation of the piecewise segment parameters and may also allow for the reduction in parameter tables, which is another advantage in the implementation of the present process. One of ordinary skill in the art will recognize that other start/end error values may be used and indeed are used in the prior art, most commonly obtained by a least squares fitting of the linear approximation to the exact values of the arrival time curve.

One of ordinary skill in the art may recognize that the smaller the value of the maximum acceptable error A, the smaller the segments' lengths nⱼ-nᵢ become. Referring again to FIG. 3a, one of ordinary skill in the art also recognizes that segments will be shorter at the initial part of the curve along n where the slope S varies rapidly, and will be longer at the deeper part of the curve where the slope S is almost constant. As understood by one of ordinary skill in the art, it is common practice in ultrasound imaging to begin adding an element's contribution to the beam starting from a non-zero depth n_{f}=2f|x|, where f is typically chosen in the range 1 to 3, and |x| is the element's distance to the center element 22. Generally, when the value of f>1.4 and the maximum error Δ=1/32 F_{c}, eight (8) segments may be sufficient to approximate the tₙ curve over the whole depth range commonly used. One of ordinary skill in the art recognizes that when utilizing probes other than phased arrays, such as linear or curvilinear arrays, additional segments, e.g, twelve (12), are required to obtain the same delay accuracy as achieved herein. However, linear and curvilinear arrays have significantly smaller parameter tables and may not require application of the process of the present invention. In some phased array applications, it may be desirable to obtain smaller error values and/or to use smaller values of f, in which case the number of segments is greater than eight (8) as is understood by one of ordinary skill in the art.

As a result, the piecewise linear approximation of arrival time may be expressed in the first algorithm of Formula (7) as follows:
First Algorithm (7)
For each beam direction and each element

1. Parameter calculation
calculate the starting depth n_{f}=integer(2f|x|).
calculate the starting arrival time tₙ₁=t_{nf}−Δ and save in table
choose n_{f} as the start n₁ of the first segment.
search for the largest n₂ for which the absolute value of the linear approximation error for all points between n₁ and n₂ is less than or equal to Δ and save that value as start of the next segment. The linear approximation is based on choosing a value of tₙ₂ smaller by Δ than the exact one. Save the last slope used as slope S₁₂ of first segment.
repeat the previous step for all segments but last. For the last segment a fixed slope S₈₉≅1 can be used and does not need to be saved

2. Arrival time calculation (prior art technique)
initialize arrival time accumulator to tₙ₁
initialize increment register to S₁₂
at beam transmit time start counting until depth n₁ then start calculation
add increment to accumulator (n₂-n₁) times, then update increment register to next segment's slope
repeat the previous step until last segment or the end of the depth range

Referring to the parameter calculation of the first algorithm, these steps may be performed prior to engaging the beamformer and beginning the beamforming operation. Referring to the arrival time calculation of the first algorithm, these steps may be performed in real-time in each beamformer channel by a circuit, e.g., the delay controller circuit 40 of FIG. 4, as is known to one of ordinary skill in the art, used to implement the delay controller 16 of FIG. 1a of the channels 11 of FIG. 1. The delay controller circuit 40 may include a slope memory 41, a segment start memory 42, a depth counter 43, a depth comparator 44, an adder 45, an arrival time accumulator 46 and a controller 47. Before starting the arrival time calculation, the slope memory 41 and segment start memory 42 may be loaded with the respective parameters, the arrival time accumulator 46 may be initialized by the initialization controller 13 shown in FIG. 1, and the depth counter may be reset. Controller 47 may select the first segment start from the parameter table and, upon receiving the start of beam signal from the beamformer, the controller 47 may enable the depth counter 43. When the depth counter 43 reaches the first segment start, the depth comparator 44 may detect the first segment start, which may then signal the first segment start to the controller 47. The controller 47 may select the first slope and enable the adder 45 to begin incrementing the arrival time accumulator 46. At the same time the second segment start may be selected to allow the depth counter 43 and depth comparator 44 to detect the end of the first segment. The process repeats for all segments until reaching the end of the beam when the initialization controller 13 may begin initializing for the next beam. Often this circuit is modified as known to one of ordinary skill in the art by storing segment lengths instead of segment starts, loading the segment lengths into the depth counter 43, which then becomes a downcounter, and replacing the depth comparator 44 with a zero detector. The process of the present invention is equally applicable to such modifications and other modifications too, because the segment lengths are merely the differences of consecutive segment starts as understood by one of ordinary skill in the art.

From the aforementioned discussion, the delay controller circuit 40 requires a parameter table containing sixteen (16) parameters for each beam direction and each aperture element in order to implement the delay generator. Generally, the parameter table requires the following: eight (8) segment start depths nᵢ, i=1, 2 . . . 8, an initial arrival time tₙ₁, and seven (7) slopes where Sᵢⱼ (j=i+1). The eighth slope value is fixed and does not have to be stored for each beam and element separately.

In order to reduce the size of the parameters table we choose for each element segment end points (kᵢ) and maximum errors (δ) proportional to the element's distance from the beam origin x in Equations (8) and (9) respectively as follows:
nᵢ=|x|×kᵢ (8)
Δ=|x|×δ (9)
where kᵢ and δ are equivalent for all array elements to the left and all array elements to the right of the beam's origin.

With this choice we obtain the following values for the initial arrival time estimate (Equation 10), the slopes (Equation 11) and the approximation error at depth n (Equation 12) as follows:

$$\begin{matrix}{\hat{t}}_{n 1} = \vert x \vert ( \frac{2 f + \sqrt{4 {f}^{2} + 1 - 4 f sign ( x ) sin θ}}{2} - δ ) & ( 10 ) \\ {S}_{ij} = 0.5 + \frac{\sqrt{{k}_{j}^{2} + 1 - 2 {k}_{j} sign ( x ) sin θ} - \sqrt{{k}_{i}^{2} + 1 - 2 {k}_{i} sign ( x ) sin θ}}{2 ( {k}_{j} - {k}_{i} )} & ( 11 ) \\ {ɛ}_{n} = \vert x \vert ( \frac{{k}_{i} \sqrt{{k}_{i}^{2} + 1 - 2 {k}_{i} sign ( x ) sin θ}}{2} - δ + \frac{{k}_{n} - {k}_{i}}{{k}_{j} - {k}_{i}} {S}_{ij} - & ( 12 ) \\ \frac{{k}_{n} + \sqrt{{k}_{n}^{2} + 1 - 2 {k}_{n} sign ( x ) sin θ}}{2} ) & \end{matrix}$$

where kₙ=n/|x|.

In Equation (10), the initial arrival time estimate is proportional to the element's distance from the beam origin. In Equation (11), the slope of segment i is the same for all elements with the same sign(x), that is, all elements to the left and all elements to the right of the beam origin. Therefore, only two sets of slopes need to be stored, a first set of slopes for the left elements and a second set of slopes for the right elements. According to Equation (12), it is observed that the estimation error is proportional with the array element's distance from the beam origin.

Due to the proportionality with 1×1 as shown in Equations (8) and (10), the parameter table requires only one set of segment starts and one initial arrival time for each of the left side and right side, and the respective quantities for all other array elements on the same side can be calculated according to the following proportionality principle of Equations (13) and (14):

$$\begin{matrix}{n}_{i}^{x 2} = \frac{\vert {x}_{2} \vert}{\vert {x}_{1} \vert} {n}_{i}^{x 1} & ( 13 ) \\ {\hat{t}}_{n 1}^{x 2} = \frac{\vert {x}_{2} \vert}{\vert {x}_{1} \vert} {\hat{t}}_{n 1}^{x 1} & ( 14 )\end{matrix}$$

where the superscript represents the distance x from the center of the element to the beam origin.

When the beam origin is located halfway between two elements then for the first left and right elements next to the beam origin we have according to Equation (15):
|x₁|=0.5 pitch (15)
where the pitch is the distance between the centers of two adjacent elements, and for any element e>1 further away in the left or right direction from the origin we have according to Equation (16):
|xₑ|=(2e−1)|x₁| (16)

Substituting Equation (16) in Equations (13) and (14) and choosing the first left/right element next to the beam origin to calculate the initial n and t parameters we obtain the following Equations (17) and (18):
nᵢ^{e}=(2e−1)nᵢ¹ (17)
\hat{t}ₙ₁^{e}=(2e−1)\hat{t}ₙ₁¹ (18)
where the superscript represents the element number as counted from the beam origin.

The relationships expressed in Equations (17) and (18) provide a convenient way to calculate all left and right elements' parameters starting from the first left elements' and first right element's parameters. When a multiplier is utilized these relationships expressed in Equations (17) and (18) may be used directly. If multipliers are not utilized, then the parameters may be calculated iteratively, from element to element, by the relationships expressed in Equations (17′) and (18′):
nᵢ^{e}=nᵢ^{e-1}+2nᵢ¹ (17′)
\hat{t}ₙ₁^{e}=\hat{t}ₙ₁^{e-1}+2tₙ₁¹ (18′)

In accordance with these relationships, the parameter tables may be reduced to 32 parameters per beam direction, 16 for each first left element and for each first right element, that is, eight (8) segment start depths for each first left element and first right element, an initial arrival time and seven (7) slopes, or as expressed when using beam symmetry, 2 Kwords for 128 beams. A 64-fold reduction relative to the processes and systems of the prior art is accomplished by implementing the process and system of the present invention. By assigning the same segment start points for the left elements and right elements which are symmetric with respect to the center element 22, another quarter of the parameters may be eliminated. In the previous example, assigning the same segment start points for the left elements and right elements would result in a memory reduction from 2 Kwords to 1.5 Kwords.

If the beam origin is not halfway between the array elements then relationships (16) to (18′) are no longer valid. However, the process based upon the proportionality relationships (13), (14) is still applicable. We may substitute increments 2nᵢ¹ and 2\hat{t}ₙ₁¹ with incr(nᵢ), incr(\hat{t}ₙᵢ), respectively, in Equations (17′), (18′) and derive the following Equations (17″), (18″):
nᵢ^{e}=nᵢ^{e-1}+incr(nᵢ) (17″)
\hat{t}ₙ₁^{e}=\hat{t}ₙ₁^{e-1}+incr(\hat{t}ₙ₁) (18″)

Note that in these equations the variables are different on the left and right side. Note that the symbol incr( ) in equations (17″), (18″) refers to the increment of the respective quantity from element to element. Equations (17″), (18″) may also be formulated using multiplications and initial values as is understood by one of ordinary skill in the art. However, the use of multiplications and initial values yields less reduction in parameter table size since both initial values for the first left elements and first right elements and increments from a first element to second element have to be provided for both the initial arrival time parameter and segment start parameter. The need to store the increments may be avoided if the beam origin distance from the closest element center is obtained by integer division of the pitch, because the increments may then be obtained by an integer multiple of left and right initialization parameters stored in the parameter table.

Referring again to the first algorithm and based upon the relationships discussed above, the parameters calculation (step 1) of the first algorithm may be modified such that the parameter calculations for each beam direction and each element may be carried out in two (2) sub-steps.

In sub-step 1.1, the calculation of parameters for the first left element and first right element by the procedure outlined in the first algorithm description except that instead of the desired maximum error Δₘₐₓ a smaller value δ=Δₘₐₓ/(numberOfElements−1) is used. For reasons of numerical accuracy it is often preferable to do the calculations for an element e>1, where e represents the number of the element counting from the center of the aperture, using a maximum error (2e−1)δ and then to obtain the parameter for the first left/right elements by division by (2e−1). In this case the search for the segment end has to be performed in steps of (2e−1) samples such that the corresponding values for the first left/right elements be integers. Sub-step 1.1 takes place off-line and results in the parameter tables used to initialize the beamformer for a particular probe.

In sub-step 1.2, calculation of parameters for all left and all right elements using equations (17), (18) or (17′), (18′), or their equivalents for beams originating at aperture positions other than halfway between elements. Sub-step 1.2 takes place in real time, before the start of each beam and is performed by the initialization controller 13 of the beamformer 1.

An example of the modified algorithm (7′) is described below, where for simplicity it is assumed that the origin of the beam is at the geometrical center of the aperture.
Modified First Algorithm (7′)
For each beam direction

1. Parameter calculation
1.1. For first left and first right elements—use e'th left/right elements
calculate the starting depth n_{f}=closeset integer multiple of (2e−1) to 2f|xₑ|.
calculate the starting arrival time tₙ₁=t_{nf}−(2e−1)δ and save in table
choose n_{f} as the start n₁ of the first segment.
Search starting from n₁ and advancing in steps of (2e−1) for the largest n₂ for which the absolute value of the linear approximation error is less than or equal to (2e−1)δ and save that value as start of the next segment. The linear approximation is based on choosing a value of tₙ₂ smaller by (2e−1)δ than the exact one Save the last slope used as slope S₁₂ of first segment.
repeat the previous step for all segments but last. For the last segment a fixed slope S₈₉=1 can be used and does not need to be saved
divide all segment start values by (2e−1)
1.2. Parameter calculation for each element further from the center than the first left or the first right element
   This sub-step if performed in real-time, iteratively from element to element, before each beam start and is described in detail in the following paragraphs.

2. Arrival time calculation (prior art technique)—for each element
initialize arrival time accumulator to tₙ₁
initialize increment register to S₁₂
at beam transmit time start counting until depth n₁ then start calculation
add increment to accumulator (n₂-n₁) times, then update increment register to next segment's slope
repeat the previous step until last segment or the end of the depth range

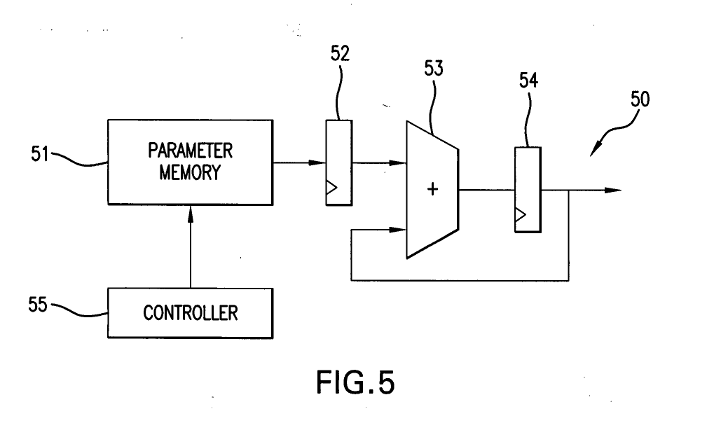

Referring now to FIGS. 1, 1a, 4 and 5, a representative circuit for use with the initialization controller 13 of FIG. 1 is shown in FIG. 5. An initialization controller circuit 50 represents only one of many different embodiments of such a circuit. The initialization controller circuit 50 may comprise a parameter memory 51, a register 52, an adder 53, an accumulator register 54 and a controller 55. For purposes of explanation and not to be taken in a limiting sense, the implementation of the initialization controller circuit 50 may be described using Equations (17′), (18′) when the beam origin is located halfway between two elements. The parameter memory 51 may be a read-only or write/read memory containing the reduced-size parameter table for the first left elements and first right elements in accordance with the present invention. The delay controllers' initialization takes place in the following sequence. The controller 55 receives the next beam's characteristics, e.g., steering angle, and selects the respective parameters block in the parameter memory 51.

The left slopes are read from the parameter memory 51 and each slope is written to the slope memory 41 of each delay controller 40 associated with each left element.

The same operation may be repeated for each right element. Referring again to FIG. 5, the initial arrival time (t(1)) for the first left element is read from the parameter memory 51 into register 52 and from there into accumulator 54 via the adder 53 which may be controlled to pass the value unmodified. Subsequently, the register 52 may shift the value up by 1 bit to obtain the left initial arrival time increment. Referring again to FIG. 4, the contents of accumulator 54 may be used to initialize register 46 of the delay controller 40 associated with the first left element. Once the register 46 is initialized, the register 52 is added to the accumulator 54 and the result is used to initialize the register 46 of the delay controller associated with the next left element. These operations repeat until all registers 54 for the arrival times of the left elements are initialized. Once the registers 54 for the arrival times of the left elements are initialized, the registers 54 for the arrival times of the right elements are initialized using the same procedures outlined for the accumulator registers of the left elements. The segment starts for the left elements and right elements are then initialized with sequences similar to the ones for the initial values of the arrival times of the registers 54.

As is understood by one of ordinary skill in the art, conventional receive beamformers employ a control signal having a delay (r−d) corrected by an additive constant to obtain positive delay numbers on all channels as shown in Equation (19):
delay=K−(d−r)/c (19)
One of ordinary skill in the art will recognize that all the teachings applicable to and equations derived for (d+r) are also applicable, after changing the sign of some terms, to the (d−r) portion of the delay as shown in Equation (19). The difference being that in the region of interest the curve (d−r) is decreasing with depth. Therefore, one of ordinary skill in the art can easily adapt the algorithms and circuits described herein for arrival time to determine delay calculation as well.

Finally, one of ordinary skill in the art will also recognize that in many practical implementations conventional receive beamformers are partitioned into multiple sections, e.g., one subsection per printed circuit board. Each section handles a subset of channels; each channel may have its own initialization controller 13. The process of the present invention can also be used in these applications, with small modifications which will be obvious to one of ordinary skill in the art.

One or more embodiments of the present invention have been described. Nevertheless, it will be understood that various modifications may be made without departing from the spirit and scope of the invention. Accordingly, other embodiments are within the scope of the following claims.

What is claimed is:

1. In an ultrasound imaging system comprising an ultrasound transducer having an array of elements and a beam origin located between two adjacent elements, each element for converting received energy into an echo signal, a process for calculating initialization parameters from a reduced parameter table, the process comprising the steps of:
generating a piecewise linear approximation of a timing function of a reflected ultrasound beam for a first left element located to the left of a beam origin and a first right element located to the right of said beam origin;
generating at least one initialization parameter comprising an initial time, an integer-valued segment start, an integer-valued segment end and a slope of a segment for said piecewise linear approximation for said first left element and said first right element;
selecting a maximum acceptable error proportional to a distance measured from said beam origin to a first left center of said first left element or to a first right center of said first right element; and
generating at least one next initialization parameter comprising a next initial time, a next integer-valued segment start, a next integer-valued segment end, and a next slope of at least one next segment for said piecewise linear approximation for at least one adjacent left element to the left of said first left element and at least one adjacent right element to the right of said first right element.

2. The process of claim 1, further comprising storing in a memory device of said ultrasound system the following: said integer-valued segment start of said segment for said first left element for at least one beam direction and said first right element for at least one beam direction, said slope for said segment for said first left element for said at least one beam direction and said first right element for said at least one beam direction; and said initial time of said segment for said first left element for said at least one beam direction and said first right element for said at least one beam direction.

3. The process of claim 2, wherein the storing step comprises the following steps:
storing eight integer-valued segment starts of said segment for said first left element and said first right element;
storing seven slopes for said segment for said first left element and said first right element; and
storing one initial time for said segment of said first left element and said first right element.

4. The process of claim 1, wherein said piecewise linear approximation at said integer-valued segment start and at said integer-valued segment end are less than said timing function by said maximum acceptable error.

5. The process of claim 4, wherein said initial time, said integer-valued segment start, and said maximum acceptable error are proportional to a distance from said beam origin to said first left center and to said first right center.

6. The process of claim 5, wherein said first left element and said at least one adjacent left element each comprise an equivalent amount of said segments and said at least one next segment and wherein said first right element and said at least one adjacent right element each comprise an equivalent amount of said segments and said at least one next segment.

7. The process of claim 5, wherein said segment and said at least one next segment each comprise an equivalent slope.

8. The process of claim 5, wherein the generation of said at least one next initialization parameter comprises the steps of:
generating a next initial time of said at least one left element by multiplying a distance measured from a left center of said at least one left element to said beam origin by an initial time of a reference left element that is stored in a memory device of said ultrasound system;
generating a next integer-valued segment start of said at least one left element by multiplying said distance measured from said left center of said at least one left element to said beam origin by an integer-valued segment start of said reference left element that is stored in said memory device; and
generating a next integer-valued segment end of said at least one left element by multiplying said distance measured from said left center of said at least one left element to said beam origin by an integer-valued segment end of said reference left element that is stored in said memory device.

9. The process of claim 5, wherein the generation of said at least one next initialization parameter comprises the steps of:
generating a next initial time of said at least one right element by multiplying a distance measured from a right center of said at least one right element to said beam origin by an initial time of a reference right element that is stored in a memory device of said ultrasound system;
generating a next integer-valued segment start of said at least one right element by multiplying said distance measured from said right center of said at least one right element to said beam origin by an integer-valued segment start of said reference right element that is stored in said memory device; and
generating a next integer-valued segment end of said at least one right element by multiplying said distance measured from said right center of said at least one right element to said beam origin by an integer-valued segment end of said reference right element that is stored in said memory device.

10. The process of claim 5, further comprising calculating a first distance from said beam origin to said first left element and calculating a second distance from said beam origin to said first right element, wherein said first distance and said second distance are equivalent.

11. The process of claim 10, wherein the generation of said at least one next initialization parameter comprises the steps of:
generating a next initial time of said at least one adjacent left element by adding an initial time of a prior adjacent left element and said initial time of said first left element multiplied by two that is stored in a memory device of said ultrasound system;
generating a next integer-valued segment start of said at least one adjacent left element by adding an integer-valued segment start of a prior adjacent element and said integer-valued segment start of said first left element multiplied by two that is stored in said memory device; and
generating a next integer-valued segment end of said at least one adjacent left element by adding an integer-valued segment end of said prior adjacent left element and said integer-valued segment end of said first left element multiplied by two that is stored in said memory device.

12. The process of claim 10, wherein the generation of said at least one next initialization parameter comprises the steps of:
generating a next initial time of said at least one adjacent right element by adding an initial time of a prior adjacent right element and said initial time of said first right element multiplied by two that is stored in said memory device;
generating a next integer-valued segment start of said at least one adjacent right element by adding an integer-valued segment start of said prior adjacent right element and said integer-valued segment start of said first right element multiplied by two that is stored in said memory device; and
generating a next integer-valued segment end of said at least one adjacent right element by adding an integer-valued segment end of said prior adjacent right element and said integer-valued segment end of said first right element multiplied by two that is stored in said memory device.

13. The process of claim 5, wherein the generation of said at least one next initialization parameter comprises the steps of:
generating a next initial time of said at least one adjacent left element by adding an initial time of a prior adjacent left element and an initial time increment that is stored in a memory device of said ultrasound system;
generating a next integer-valued segment start of said at least one adjacent left element by adding an integer-valued segment start of said prior adjacent left element and a next integer-valued segment start increment that is stored in said memory device; and
generating a next integer-valued segment end of said at least one adjacent left element by an integer-valued segment end of a prior adjacent left element and an integer-valued segment end increment that is stored in said memory device.

14. The process of claim 5, wherein the generation of said at least one next initialization parameter comprises the steps of:
generating a next initial time of said at least one adjacent right element by adding an initial time of a prior adjacent right element and an initial time increment that is stored in a memory device of said ultrasound system;
generating a next integer-valued segment start of said at least one adjacent right element by adding an integer-valued segment start of said prior adjacent right element and a next integer-valued segment start increment that is stored in said memory device; and
generating a next integer-valued segment end of said at least one adjacent right element by an integer-valued segment end of a prior adjacent right element and an integer-valued segment end increment that is stored in said memory device.

15. The process as in any of claims 11-14, wherein said initial time of said prior adjacent left element is said initial time of said first left element or said next initial time of said at least one adjacent left element.

16. The process as in any of claims 11-14, wherein said integer-valued segment start of said prior adjacent left element is said integer-valued segment start of said first left element or said next integer-valued segment start of said at least one adjacent left element.

17. The process as in any of claims 11-15, wherein said integer-valued segment end of said prior adjacent left element is said integer-valued segment end of said first left element or said next integer-valued segment end of said at least one adjacent left element.

18. The process as in any of claims 11-14, wherein said initial time of said prior adjacent right element is said initial time of said first right element or said next initial time of said at least one adjacent right element.

19. The process as in any of claims 11-14, wherein said integer-valued segment start of said prior adjacent right element is said integer-valued segment start of said first right element or said next integer-valued segment start of said at least one adjacent right element.

20. The process as in any of claims 11-14, wherein said integer-valued segment end of said prior adjacent right element is said integer-valued segment end of said first right element or said next integer-valued segment end of said at least one adjacent right element.

21. An ultrasound system, comprising:
an ultrasound transducer comprising an array of elements and a beam origin located between two adjacent elements, each of said elements for converting received energy into an echo signal; and
a beamformer comprising:
an initialization controller including initialization controller circuitry for calculating initialization parameters according to the process of claims 1-20;
at least one channel including delay circuitry and tracking circuitry for tracking an ultrasound beam; and
a summer for summing phase-aligned signals to form a beamformed signal.

22. The ultrasound system of claim 21, wherein said initialization controller circuitry comprises:
a parameter memory for use with said reduced parameter table comprising at least one initialization value for at least one element to the left of a center of an aperture and at least one element to the right of said center of said aperture;
a first register for reading said at least one initialization value from said parameter memory and for shifting said at least one initialization value to generate said at least one initialization value increment;
an adder for receiving said at least one initialization value from said register;
a second register for receiving and accumulating said at least one initialization value from said adder; and
a controller for selecting said at least one initialization value from said parameter memory.

23. The ultrasound system of claim 22, wherein said adder receives said at least one initialization value in an unmodified condition.

24. The ultrasound system of claim 21, wherein said reduced parameter table comprises the following initialization parameters: an integer-valued segment start of a segment for a first left element for at least one beam direction and a first right element for at least one beam direction, a slope for said segment for said first left element for said at least one beam direction and said first right element for said at least one beam direction; and an initial time of said segment for said first left element for said at least one beam direction and said first right element for said at least one beam direction.
